(19) United States
(12) Patent Application Publication
(10) Pub. No.: US 2020/0178840 A1
(43) Pub. Date: Jun. 11, 2020

(54) METHOD AND DEVICE FOR MARKING ADVENTITIOUS SOUNDS

(71) Applicant: Industrial Technology Research Institute, Hsinchu (TW)

(72) Inventors: Chun-Fu YEH, Xingang Township (TW); Yi-Fei LUO, Zhudong Township (TW); Cheng-Li CHANG, Hsinchu City (TW); I-Ju YEH, Hsinchu City (TW)

(73) Assignee: Industrial Technology Research Institute, Hsinchu (TW)

(21) Appl. No.: 16/235,546

(22) Filed: Dec. 28, 2018

(30) Foreign Application Priority Data
Dec. 6, 2018 (TW) 107143834

(51) Int. Cl. A61B 5/08; A61B 5/00; G06N 3/04; G06N 3/08; G06N 20/00

(52) U.S. Cl. CPC A61B 5/08; A61B 5/7267; G06N 20/00; A61B 5/7225; G06N 3/08; G06N 3/0481

(57) ABSTRACT

A method for marking adventitious sounds is provided. The method includes: receiving a lung sound signal generated by a sensor from a chest cavity sound signal; capturing a lung sound signal segment from the lung sound signal every sampling time interval; converting the lung sound signal segments into spectrograms; inputting the spectrograms into a recognition model to determine whether the spectrograms include adventitious sounds; obtaining time points of occurrence corresponding to the adventitious sounds according to abnormal spectrograms including the adventitious sounds, and the number of occurrences of the adventitious sounds corresponding to the time points; and marking an adventitious sound signal segment having the highest probability of occurrence of the adventitious sound in the lung sound signal according to the time points and the number of occurrences.

### CROSS REFERENCE TO RELATED APPLICATIONS

This application claims priority from Taiwan Patent Application No. 107143834, filed on Dec. 6, 2018, the disclosure of which is incorporated herein by reference in its entirety.

### BACKGROUND

### Technical Field

The disclosure relates to biological recognition technology, and more particularly, it relates to a method and a device for marking adventitious sounds.

### Description of the Related Art

In recent years, Chronic Obstructive Pulmonary Disease (COPD) has become one of the top ten causes of death in the world. At present, the diagnosis of COPD patients still needs to be auscultated by experienced clinicians. Clinicians make a diagnosis through a medical record filled out by a patient and auscultation results. However, this auscultation method does not provide complete information as a reference for clinicians.

Therefore, a method and a device for marking adventitious sounds are desired to detect a large amount of lung sound signals provided by COPD patients and mark the time points at which the adventitious sound signals occur for clinicians to determine the patient's condition.

### SUMMARY

The following summary is illustrative only and is not intended to be limiting in any way. That is, the following summary is provided to introduce concepts, highlights, benefits and advantages of the novel and non-obvious techniques described herein. Selected, not all, implementations are described further in the detailed description below. Thus, the following summary is not intended to identify essential features of the claimed subject matter, nor is it intended for use in determining the scope of the claimed subject matter.

A method and a device for marking adventitious sounds are provided in the disclosure.

In an embodiment, a method for marking adventitious sounds is provided in the disclosure. The method comprises: receiving a lung sound signal generated by a sensor from a chest cavity sound signal; capturing a lung sound signal segment from the lung sound signal every sampling time interval; converting the lung sound signal segments into spectrograms; inputting the spectrograms into a recognition model to determine whether the spectrograms include adventitious sounds; obtaining time points of occurrence corresponding to the adventitious sounds according to abnormal spectrograms including the adventitious sounds, and the number of occurrences of the adventitious sounds corresponding to the time points; and marking an adventitious sound signal segment having the highest probability of occurrence of the adventitious sound in the lung sound signal according to the time points and the number of occurrences.

In some embodiments, each of the lung sound signal segments has a length, and the length is greater than one breath cycle time.

In some embodiments, the step of obtaining time points of occurrence corresponding to the adventitious sounds according to abnormal spectrograms including the adventitious sounds, and the number of occurrences of the adventitious sounds corresponding to the time points further comprises: capturing a feature map from each of the abnormal spectrograms and weights corresponding to classes of the lung sounds by using the recognition model; obtaining activation maps according to the feature maps and the weights; obtaining locations where the adventitious sounds occur according to the activation maps; and obtaining the time points of occurrence corresponding to the adventitious sounds according to the locations, and computing the number of occurrences of the adventitious sounds corresponding to the time points.

In some embodiments, the sum F of the feature map m is expressed as follows:

F=Σₘfₘ(x, y)

wherein f(x, y) represents a value of the feature map at a spatial location (x, y), and the activation map MAP_{c}(x, y) for a class c of lung sound is expressed as follows:

$${MAP}_{c} ( x , y ) = {\sum }_{m} {w}_{m}^{c} fm ( x , y )$$

wherein wₘ^{c} represents a weight corresponding to the class c of lung sound of the m^{th} feature map.

In some embodiments, the step of marking an adventitious sound signal segment having the highest probability of occurrence of the adventitious sound in the lung sound signal according to the time points and the number of occurrences further comprises: counting the number of occurrences of the adventitious sounds in a time window for every predetermined time period through the time window; and selecting a first time window having the highest number of occurrences, and marking the adventitious sound signal segment in the lung sound signal according to the first time window.

In some embodiments, each of the lung sound signal segments has a length, and the length is greater than one sampling time interval.

In some embodiments, before capturing the lung sound signal segment, the method further comprises: performing band-pass filtering, pre-amplification, and pre-emphasis on the chest cavity sound signal to generate the lung sound signal.

In some embodiments, the lung sound signal segments are converted into spectrograms by the Fourier Transform.

In some embodiments, the recognition model is based on a convolutional neural network (CNN) model.

In an embodiment, a device for marking adventitious sounds is provided. The device comprises one or more processors and one or more computer storage media for storing one or more computer-readable instructions. The processor is configured to drive the computer storage media to execute the following tasks: receiving a lung sound signal generated by a sensor from a chest cavity sound signal; capturing a lung sound signal segment from the lung sound signal every sampling time interval; converting the lung sound signal segments into spectrograms; inputting the spectrograms into a recognition model to determine whether the spectrograms include adventitious sounds; obtaining time points of occurrence corresponding to the adventitious sounds according to abnormal spectrograms including the adventitious sounds, and the number of occurrences of the adventitious sounds corresponding to the time points; and marking an adventitious sound signal segment having the highest probability of occurrence of the adventitious sound in the lung sound signal according to the time points and the number of occurrences.

### BRIEF DESCRIPTION OF DRAWINGS

The accompanying drawings are included to provide a further understanding of the disclosure, and are incorporated in and constitute a part of the present disclosure. The drawings illustrate implementations of the disclosure and, together with the description, serve to explain the principles of the disclosure. It should be appreciated that the drawings are not necessarily to scale as some components may be shown out of proportion to the size in actual implementation in order to clearly illustrate the concept of the present disclosure.

The patent or application file contains at least one color drawing. Copies of this patent or patent application publication with color drawing will be provided by the USPTO upon request and payment of the necessary fee.

FIG. 1 shows a schematic diagram of a system for marking adventitious sounds according to one embodiment of the present disclosure.

FIG. 2 is a flowchart illustrating a method for marking adventitious sounds according to an embodiment of the present disclosure.

FIG. 3 illustrates a convolutional neural network according to an embodiment of the present disclosure.

FIG. 4A illustrates an activation map according to an embodiment of the present disclosure.

FIG. 4B illustrates the locations at which the adventitious sounds occur according to an embodiment of the present disclosure.

FIG. 5 is a schematic diagram illustrating a lung sound signal according to an embodiment of the present disclosure.

FIG. 6A is a schematic diagram illustrating the occurrence of adventitious sounds corresponding to each time point of occurrence according to an embodiment of the present disclosure.

FIG. 6B is a histogram illustrating the number of occurrences of adventitious sounds corresponding to each time point of occurrence according to an embodiment of the present disclosure.

FIGS. 7A˜7B are histograms illustrating the number of occurrences of the adventitious sounds corresponding to each time point according to an embodiment of the present disclosure.

FIG. 8 illustrates an exemplary operating environment for implementing embodiments of the present disclosure.

### DETAILED DESCRIPTION

Various aspects of the disclosure are described more fully below with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the disclosure disclosed herein, whether implemented independently of or combined with any other aspect of the disclosure. For example, an apparatus may be implemented or a method may be practiced using number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure disclosed herein may be embodied by one or more elements of a claim.

The word “exemplary” is used herein to mean “serving as an example, instance, or illustration.” Any aspect described herein as “exemplary” is not necessarily to be construed as preferred or advantageous over other aspects. Furthermore, like numerals refer to like elements throughout the several views, and the articles “a” and “the” includes plural references, unless otherwise specified in the description.

It should be understood that when an element is referred to as being “connected” or “coupled” to another element, it may be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion. (e.g., “between” versus “directly between”, “adjacent” versus “directly adjacent”, etc.).

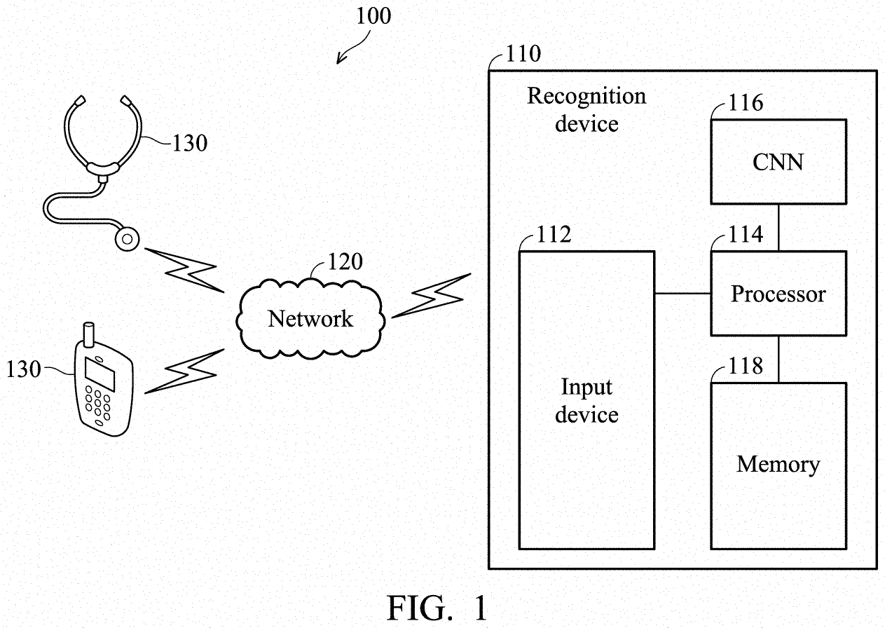

FIG. 1 shows a schematic diagram of a system 100 for marking adventitious sounds according to one embodiment of the present disclosure. The system 100 for marking adventitious sounds may include a recognition device 110 and an electronic device 130 connected to the network 120.

The recognition device 110 may include an input device 112, wherein the input device 112 is configured to receive input data from a variety of sources. For example, the recognition device 110 may receive lung sound data from the network 120 or receive lung sound signals transmitted by the electronic device 130. The recognition device 110 may receive training data including adventitious sounds, and may further be trained as a recognizer configured to recognize adventitious sounds according to the training data.

The recognition device 110 may include a processor 114, a convolutional neural network (CNN) 116 and a memory 118. In addition, the data may be stored in the memory 118 or stored in the convolutional neural network 116. In one embodiment, the convolutional neural network 116 may be implemented in the processor 114. In another embodiment, the recognition device 110 may be used with components, systems, sub-systems, and/or devices other than those that are depicted herein.

The types of recognition device 110 range from small handheld devices, such as mobile telephones and handheld computers to large mainframe systems, such as mainframe computers. Examples of handheld computers include personal digital assistants (PDAs) and notebooks. The electronic device 130 may be a device that senses the sound of the human chest, for example, a lung sound sensor or an electronic stethoscope mentioned in Taiwan Patent Application No. 107109623. The electronic device 130 may perform band-pass filtering, pre-amplification, pre-emphasis processing and the like on the sensed chest cavity sound signal to generate a lung sound signal. In one embodiment, the electronic device 130 can also be a small handheld device (e.g., a mobile phone) that receives a lung sound signal generated by a lung sound sensor or an electronic stethoscope. The electronic device 130 can transmit the lung sound signal to the recognition device 110 using the network 120. The network 120 can include, but is not limited to, one or more local area networks (LANs), and/or wide area networks (WANs). The documents listed above are hereby expressly incorporated by reference in their entirety.

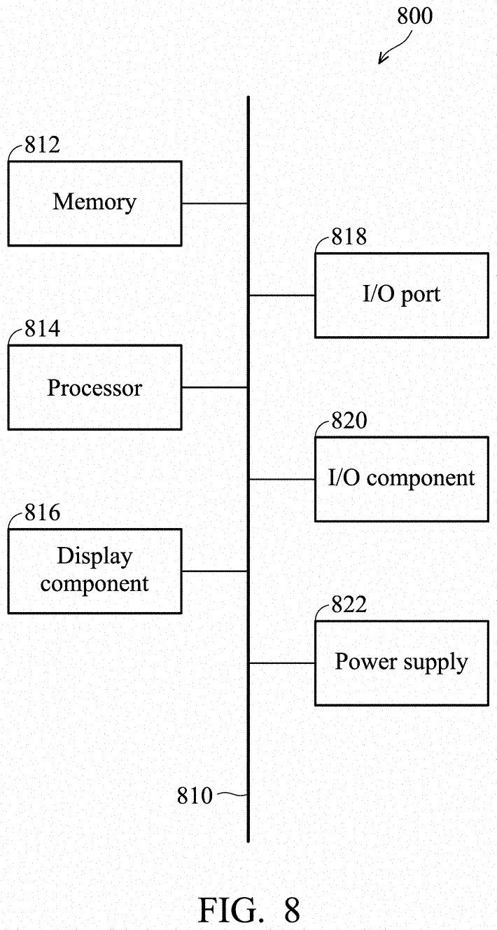

It should be understood that the recognition device 110 shown in FIG. 1 is an example of one suitable system 100 architecture marking adventitious sounds. Each of the components shown in FIG. 1 may be implemented via any type of computing device, such as the computing device 800 described with reference to FIG. 8, for example.

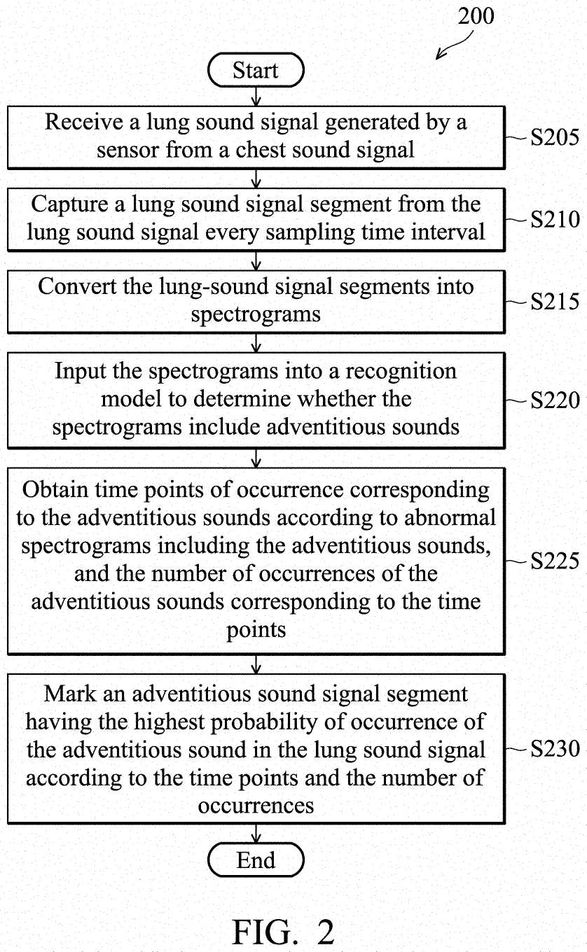

FIG. 2 is a flowchart illustrating a method 200 for marking adventitious sounds according to an embodiment of the present disclosure. The method can be implemented in the processor 114 of the recognition device 110 as shown in FIG. 1.

In step S205, the recognition device receives a lung sound signal generated by a sensor from a chest cavity sound signal. In step S210, the recognition device captures a lung sound signal segment from the lung sound signal every sampling time interval, wherein each of the lung sound signal segments has a length, and the length is greater than one breath cycle time. Then, in step S215, the recognition device converts the lung sound signal segments into spectrograms. In one embodiment, the lung sound signal segments are converted into spectrograms by the Fourier Transform.

In step S220, the recognition device inputs the spectrograms into a recognition model to determine whether the spectrograms include adventitious sounds, wherein the recognition model is based on a convolutional neural network (CNN) model and is used to recognize the classes of lung sounds of the spectrograms. In one embodiment, the classes of lung sounds may include normal sounds, wheezes, rhonchi, crackles (or rales) or other abnormal sounds. Next, in step S225, the recognition device obtains time points of occurrence corresponding to the adventitious sounds, namely time points at which adventitious sounds occur, according to abnormal spectrograms including the adventitious sounds, and the number of occurrences of the adventitious sounds corresponding to the time points. In step S230, the recognition device marks an adventitious sound signal segment having the highest probability of occurrence of the adventitious sound in the lung sound signal according to the time points and the number of occurrences.

The following may explain in detail how the recognition device obtains time points of occurrence corresponding to the adventitious sounds according to abnormal spectrograms including the adventitious sounds, and the number of occurrences of the adventitious sounds corresponding to the time points in step S225.

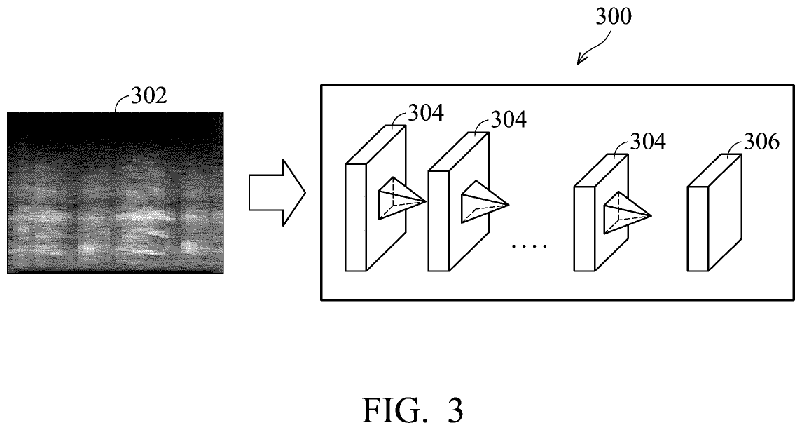

First, the recognition device captures a feature map from each of the abnormal spectrograms and weights corresponding to the classes of the lung sounds by using the recognition model based on a convolutional neural network. FIG. 3 illustrates a convolutional neural network 300 according to an embodiment of the present disclosure.

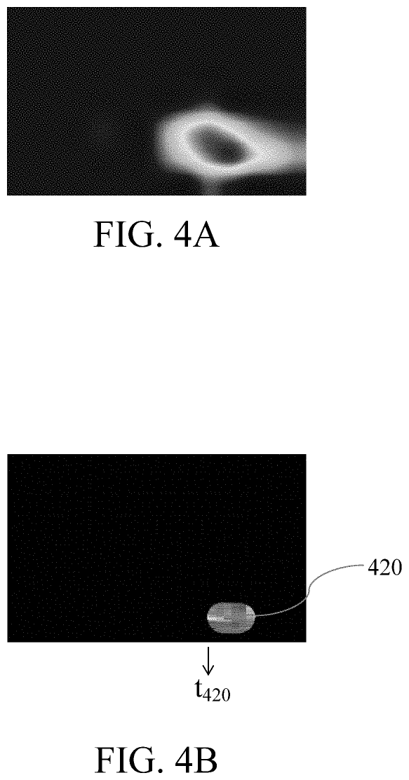

As shown in FIG. 3, the convolutional neural network 300 receives a spectrogram and through a series of applied layers, generates output. In particular, the convolutional neural network 300 utilizes a plurality of convolution layers 304, a plurality of pooling layers (not shown in FIG. 3), and a global average pooling (GAP) layer 306. Utilizing these layers, the convolutional neural network 300 generates the output. As shown in FIG. 3, the GAP layer 306 outputs the spatial average values of the feature map of each unit at the last convolutional layer. A weighted sum of these spatial average values is used to generate the final output. Similarly, a weighted sum of the feature maps of the last convolutional layer is computed to obtain an activation map 410, as shown in FIG. 4A.

Specifically, according to the convolutional neural network 300, for the m^{th} feature map on the last convolutional layer, the output of the GAP layer is defined as

F=Σₘfₘ(x,y)

wherein f(x, y) represents the value of the feature map of the m^{th} feature map at a spatial location (x, y) on the last convolutional layer. For the class c of lung sound, the activation map MAP_{c}(x, y) may be expressed as follows:

$${MAP}_{c} ( x , y ) = {\sum }_{m} {w}_{m}^{c} fm ( x , y )$$

wherein wₘ^{c} represents a weight corresponding to the class c of lung sound of the m^{th} feature map.

After obtaining the activation maps, the recognition device compares each pixel in each activation map with a first threshold. When there is a region in which the pixels are higher than the first threshold, the recognition device determines that the region is the location at which the adventitious sound occurs. As shown in FIG. 4B, the region 420 is the location where the adventitious sound occurs. The recognition device marks a time point t₄₂₀ of occurrence corresponding to the location according to the location.

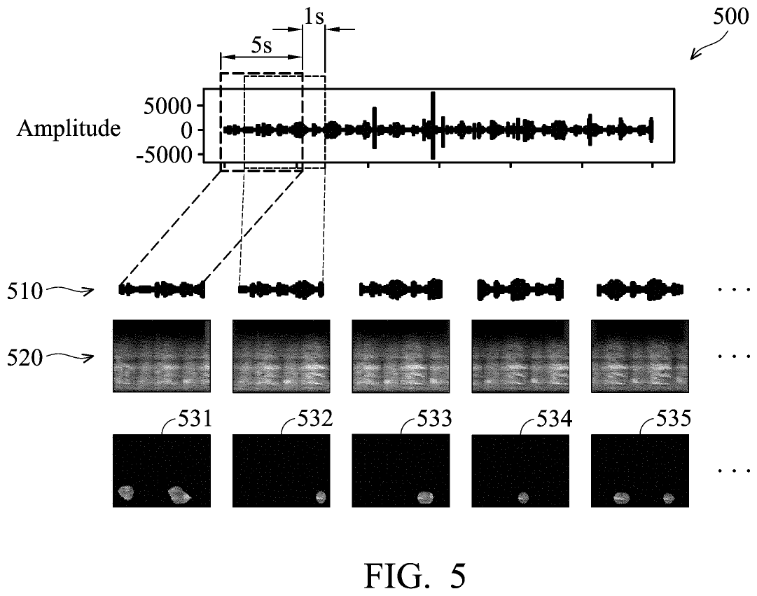

FIG. 5 is a schematic diagram illustrating a lung sound signal according to an embodiment of the present disclosure. As shown in FIG. 5, the recognition device captures a lung sound signal segment with a length of 5 seconds from the lung sound signal 500 every sampling time interval of one second. Each of the lung sound signal segments 510 is converted into spectrograms 520 by the Fourier Transform. The recognition device then uses the recognition model to find the locations of the adventitious sounds in the spectrograms 520 (as indicated by the red regions in FIGS. 531˜535), and the time points of occurrence corresponding to the locations.

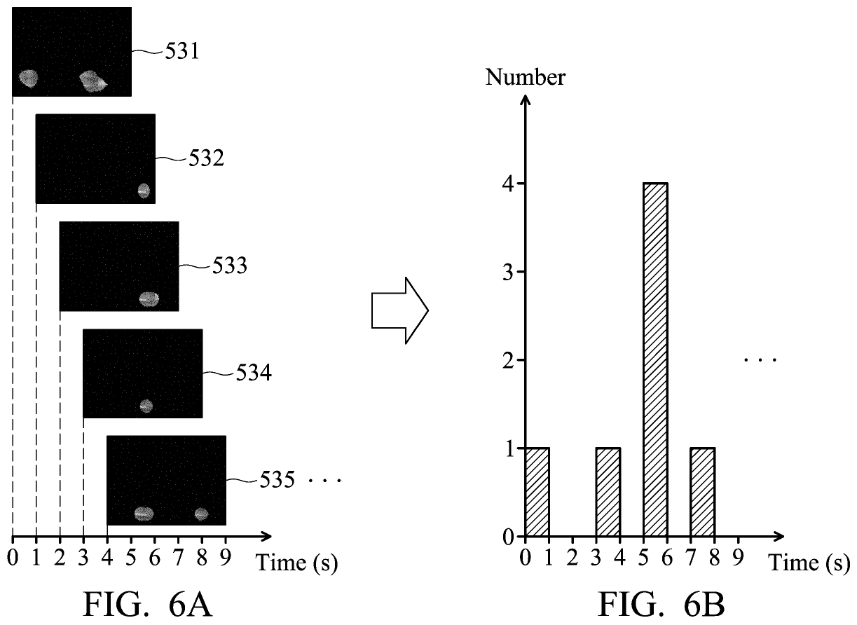

The recognition device may obtain the time points of occurrence corresponding to the adventitious sounds from the spectrograms including the adventitious sounds according to the locations, and calculates the number of occurrences of the adventitious sounds corresponding to each of the time points of occurrence. For example, the FIGS. 531 to 535 in FIG. 5 are arranged according to time to obtain FIG. 6A. The sampling time interval between two consecutive figures is 1 second, and the adventitious sounds occur at 0, 3, 5, and 7 seconds. The recognition device calculates the number of occurrences of the adventitious sounds at 0, 3, 5, and 7 seconds in the spectrograms. The histogram in FIG. 6B shows the number of occurrences of the adventitious sounds corresponding to each of the time points of occurrence, 0 to 8 seconds. As shown in FIG. 6B, the numbers of adventitious sounds occurred at the time points of occurrence, 0^{th}, 3^{rd} and 7^{th} seconds, are once, respectively, the number of adventitious sounds occurred at the time point of occurrence, 5^{th} second, is four times, and the numbers of adventitious sounds occurred at the time points of occurrence, 1^{st}, 2^{nd}, 4^{th}, 6^{th} and 8^{th} seconds, are zero, respectively.

Next, the following may explain in detail how the recognition device marks an adventitious sound signal segment having the highest probability of occurrence of the adventitious sound in the lung sound signal according to the time points and the number of occurrences in step S230.

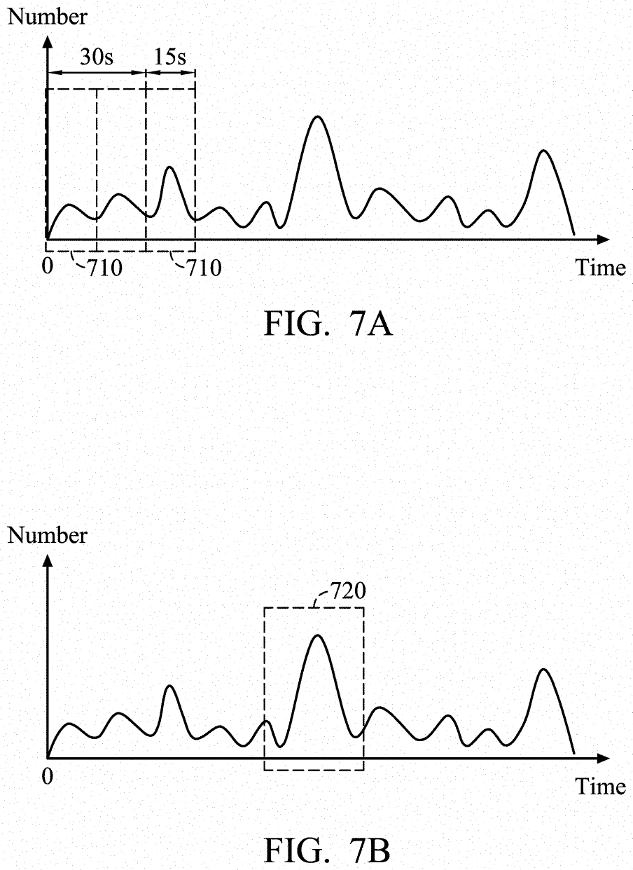

FIGS. 7A˜7B are histograms illustrating the number of occurrences of the adventitious sounds corresponding to each time point according to an embodiment of the present disclosure. The recognition device counts the number of occurrences of the adventitious sounds in a time window for every predetermined time period through the time window. In an embodiment, the length of the time window is greater than the predetermined time period. As shown in FIG. 7A, the recognition device counts the number of occurrences of the adventitious sounds in a time window for every predetermined time period of 15 seconds through the time window of 30 seconds. As shown in FIG. 7B, the recognition device selects a first time window 720 having the highest number of occurrences, and marks the adventitious sound signal segment in the lung sound signal according to the first time window 720. For example, the recognition device may obtain an adventitious sound signal segment from the lung sound signal according to the time point corresponding to the highest number of occurrences in the first time window 720, wherein the center point of the adventitious sound signal segment is the time point and the adventitious sound signal segment has a preset length. Obviously, the probability of occurrence of the adventitious sound in the adventitious sound signal segment is higher and more accurate than other segments.

As described above, the method and device for marking adventitious sounds provided in the disclosure may automatically recognize whether adventitious sounds are included in the lung sound signal, mark the time points of occurrence of adventitious sound, and obtain corresponding adventitious sound signal segments. Clinicians can use the adventitious sound signal segments to obtain information (for example, classes of adventitious sounds, time of occurrence, durations, number of occurrences, etc.) about the patient's condition, and can directly listen to the adventitious sound signal segments to save auscultation time.

Having described embodiments of the present disclosure, an exemplary operating environment in which embodiments of the present disclosure may be implemented is described below. Referring to FIG. 8, an exemplary operating environment for implementing embodiments of the present disclosure is shown and generally known as a computing device 800. The computing device 800 is merely an example of a suitable computing environment and is not intended to limit the scope of use or functionality of the disclosure. Neither should the computing device 800 be interpreted as having any dependency or requirement relating to any one or combination of components illustrated.

The disclosure may be realized by means of the computer code or machine-useable instructions, including computer-executable instructions such as program modules, being executed by a computer or other machine, such as a personal data assistant (PDA) or other handheld device. Generally, program modules may include routines, programs, objects, components, data structures, etc., and refer to code that performs particular tasks or implements particular abstract data types. The disclosure may be implemented in a variety of system configurations, including hand-held devices, consumer electronics, general-purpose computers, more specialty computing devices, etc. The disclosure may also be implemented in distributed computing environments where tasks are performed by remote-processing devices that are linked by a communication network.

With reference to FIG. 8, the computing device 800 may include a bus 810 that is directly or indirectly coupled to the following devices: one or more memories 812, one or more processors 814, one or more display components 816, one or more input/output (I/O) ports 818, one or more input/output components 820, and an illustrative power supply 822. The bus 810 may represent one or more kinds of busses (such as an address bus, data bus, or any combination thereof). Although the various blocks of FIG. 8 are shown with lines for the sake of clarity, and in reality, the boundaries of the various components are not specific. For example, the display component such as a display device may be considered an I/O component and the processor may include a memory.

The computing device 800 typically includes a variety of computer-readable media. The computer-readable media can be any available media that can be accessed by computing device 800 and includes both volatile and nonvolatile media, removable and non-removable media. By way of example, but not limitation, computer-readable media may comprise computer storage media and communication media. The computer storage media may include volatile and nonvolatile, removable and non-removable media implemented in any method or technology for storage of information such as computer-readable instructions, data structures, program modules or other data. The computer storage media may include, but not limit to, random access memory (RAM), read-only memory (ROM), electrically-erasable programmable read-only memory (EEPROM), flash memory or other memory technology, compact disc read-only memory (CD-ROM), digital versatile disks (DVD) or other optical disk storage, magnetic cassettes, magnetic tape, magnetic disk storage or other magnetic storage devices, or any other medium which can be used to store the desired information and which can be accessed by the computing device 800. The computer storage media may not comprise signal per se.

The communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, but not limitation, communication media includes wired media such as a wired network or direct-wired connection, and wireless media such as acoustic, RF, infrared and other wireless media or any combination thereof.

The memory 812 may include computer-storage media in the form of volatile and/or nonvolatile memory. The memory may be removable, non-removable, or a combination thereof. Exemplary hardware devices include solid-state memory, hard drives, optical-disc drives, etc. The computing device 800 includes one or more processors that read data from various entities such as the memory 812 or the I/O components 820. The presentation component(s) 816 present data indications to a user or other device. Exemplary presentation components include a display device, speaker, printing component, vibrating component, etc.

The I/O ports 818 allow the computing device 800 to be logically coupled to other devices including the I/O components 820, some of which may be embedded. Illustrative components include a microphone, joystick, game pad, satellite dish, scanner, printer, wireless device, etc. The I/O components 820 may provide a natural user interface (NUI) that processes gestures, voice, or other physiological inputs generated by a user. For example, inputs may be transmitted to an appropriate network element for further processing. A NUI may be implemented to realize speech recognition, touch and stylus recognition, face recognition, biometric recognition, gesture recognition both on screen and adjacent to the screen, air gestures, head and eye tracking, touch recognition associated with displays on the computing device 800, or any combination of. The computing device 800 may be equipped with depth cameras, such as stereoscopic camera systems, infrared camera systems, RGB camera systems, any combination of thereof to realize gesture detection and recognition. Furthermore, the computing device 800 may be equipped with accelerometers or gyroscopes that enable detection of motion. The output of the accelerometers or gyroscopes may be provided to the display of the computing device 800 to carry out immersive augmented reality or virtual reality.

Furthermore, the processor 814 in the computing device 800 can execute the program code in the memory 812 to perform the above-described actions and steps or other descriptions herein.

It should be understood that any specific order or hierarchy of steps in any disclosed process is an example of a sample approach. Based upon design preferences, it should be understood that the specific order or hierarchy of steps in the processes may be rearranged while remaining within the scope of the present disclosure. The accompanying method claims present elements of the various steps in a sample order, and are not meant to be limited to the specific order or hierarchy presented.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements.

While the disclosure has been described by way of example and in terms of the preferred embodiments, it should be understood that the disclosure is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for marking adventitious sounds, comprising:
receiving a lung sound signal generated by a sensor from a chest cavity sound signal;
capturing a lung sound signal segment from the lung sound signal every sampling time interval;
converting the lung sound signal segments into spectrograms;
inputting the spectrograms into a recognition model to determine whether the spectrograms include adventitious sounds;
obtaining time points of occurrence corresponding to the adventitious sounds according to abnormal spectrograms including the adventitious sounds, and the number of occurrences of the adventitious sounds corresponding to the time points; and
marking an adventitious sound signal segment having the highest probability of occurrence of the adventitious sound in the lung sound signal according to the time points and the number of occurrences.

2. The method for marking adventitious sounds claimed in claim 1, wherein each of the lung sound signal segments has a length, and the length is greater than one breath cycle time.

3. The method for marking adventitious sounds claimed in claim 1, wherein the step of obtaining time points of occurrence corresponding to the adventitious sounds according to abnormal spectrograms including the adventitious sounds, and the number of occurrences of the adventitious sounds corresponding to the time points further comprises:
capturing a feature map from each of the abnormal spectrograms and weights corresponding to classes of the lung sounds by using the recognition model;
obtaining activation maps according to the feature maps and the weights;
obtaining locations where the adventitious sounds occur according to the activation maps; and
obtaining the time points of occurrence corresponding to the adventitious sounds according to the locations, and computing the number of occurrences of the adventitious sounds corresponding to the time points.

4. The method for marking adventitious sounds claimed in claim 3, wherein the sum F of the feature map m is expressed as follows:
F=Σₘfₘ(x, y)
wherein f(x, y) represents a value of the feature map at a spatial location (x, y), and the activation map MAP_{c}(x, y) for a class c of lung sound is expressed as follows:
$${MAP}_{c} ( x , y ) = {\sum }_{m} {w}_{m}^{c} fm ( x , y )$$
wherein wₘ^{c} represents a weight corresponding to the class c of lung sound of the m^{th} feature map.

5. The method for marking adventitious sounds claimed in claim 1, wherein the step of marking an adventitious sound signal segment having the highest probability of occurrence of the adventitious sound in the lung sound signal according to the time points and the number of occurrences further comprises:
counting the number of occurrences of the adventitious sounds in a time window for every predetermined time period through the time window; and
selecting a first time window having the highest number of occurrences, and marking the adventitious sound signal segment in the lung sound signal according to the first time window.

6. The method for marking adventitious sounds claimed in claim 1, wherein each of the lung sound signal segments has a length, and the length is greater than one sampling time interval.

7. The method for marking adventitious sounds claimed in claim 1, before capturing the lung sound signal segment, the method further comprises:
performing band-pass filtering, pre-amplification, and pre-emphasis on the chest cavity sound signal to generate the lung sound signal.

8. The method for marking adventitious sounds claimed in claim 1, wherein the lung sound signal segments are converted into spectrograms by the Fourier Transform.

9. The method for marking adventitious sounds claimed in claim 1, wherein the recognition model is based on a convolutional neural network (CNN) model.

10. A device for marking adventitious sounds, comprising:
one or more processors; and
one or more computer storage media for storing one or more computer-readable instructions, wherein the processor is configured to drive the computer storage media to execute the following tasks:
receiving a lung sound signal generated by a sensor from a chest cavity sound signal;
capturing a lung sound signal segment from the lung sound signal every sampling time interval;
converting the lung sound signal segments into spectrograms;
inputting the spectrograms into a recognition model to determine whether the spectrograms include adventitious sounds;
obtaining time points of occurrence corresponding to the adventitious sounds according to abnormal spectrograms including the adventitious sounds, and the number of occurrences of the adventitious sounds corresponding to the time points; and
marking an adventitious sound signal segment having the highest probability of occurrence of the adventitious sound in the lung sound signal according to the time points and the number of occurrences.

11. The device for marking adventitious sounds as claimed in claim 10, wherein each of the lung sound signal segments has a length, and the length is greater than one breath cycle time.

12. The device for marking adventitious sounds as claimed in claim 10, wherein the step of obtaining time points of occurrence corresponding to the adventitious sounds according to abnormal spectrograms including the adventitious sounds, and the number of occurrences of the adventitious sounds corresponding to the time points executed by the processor further comprises:
capturing a feature map from each of the abnormal spectrograms and weights corresponding to classes of the lung sounds by using the recognition model;
obtaining activation maps according to the feature maps and the weights; and
obtaining locations where the adventitious sounds occur according to the activation maps;
obtaining the time points of occurrence corresponding to the adventitious sounds according to the locations, and computing the number of occurrences of the adventitious sounds corresponding to the time points.

13. The device for marking adventitious sounds as claimed in claim 12, wherein the sum F of the feature map m is expressed as follows:
F=Σₘfₘ(x, y)
wherein f(x, y) represents a value of the feature map at a spatial location (x, y), and the activation map MAP_{c}(x, y) for a class c of lung sound is expressed as follows:
$${MAP}_{c} ( x , y ) = {\sum }_{m} {w}_{m}^{c} fm ( x , y )$$
wherein wₘ^{c} represents a weight corresponding to the class c of lung sound of the m^{th} feature map.

14. The device for marking adventitious sounds as claimed in claim 10, wherein the step of marking an adventitious sound signal segment having the highest probability of occurrence of the adventitious sound in the lung sound signal according to the time points and the number of occurrences executed by the processor further comprises:
counting the number of occurrences of the adventitious sounds in a time window for every predetermined time period through the time window; and
selecting a first time window having the highest number of occurrences, and marking the adventitious sound signal segment in the lung sound signal according to the first time window.

15. The device for marking adventitious sounds as claimed in claim 10, wherein each of the lung sound signal segments has a length, and the length is greater than one sampling time interval.

16. The device for marking adventitious sounds as claimed in claim 10, before capturing the lung sound signal segment, the processor further executes the following tasks:
performing band-pass filtering, pre-amplification, and pre-emphasis on the chest cavity sound signal to generate the lung sound signal.

17. The device for marking adventitious sounds as claimed in claim 10, wherein the lung sound signal segments are converted into spectrograms by the Fourier Transform.

18. The device for marking adventitious sounds as claimed in claim 10, wherein the recognition model is based on a convolutional neural network (CNN) model.
